(19) United States
(12) Patent Application Publication
(10) Pub. No.: US 2024/0382336 A1
(43) Pub. Date: Nov. 21, 2024

(54) FLUID COLLECTION DEVICES HAVING A COLLECTION BAG, AND RELATED SYSTEMS AND METHODS

(71) Applicant: PUREWICK CORPORATION, Covington, GA (US)

(72) Inventors: Hannah Rose Kriscovich, Marietta, GA (US); Dennis Joseph, Milton, GA (US); Qihua Xu, Cary, NC (US); Juan Alejandro Saavedra Cisneros, Dacula, GA (US); Jill W. Jones, Avondale Estates, GA (US)

(21) Appl. No.: 18/662,216

(22) Filed: May 13, 2024

Related U.S. Application Data
(63) Continuation of application No. 17/657,474, filed on Mar. 31, 2022, now Pat. No. 12,029,677.
(60) Provisional application No. 63/171,165, filed on Apr. 6, 2021.

(51) Int. Cl. A61F 5/451; A61F 5/44

(52) U.S. Cl. CPC A61F 5/451; A61F 5/4401

(57) ABSTRACT

Examples relate to fluid collection devices, and related systems and methods. In some embodiments, a fluid collection device includes a fluid impermeable barrier and a fluid permeable body. The fluid impermeable barrier may include a first end region, a second end region, a front side, and a back side. The fluid impermeable barrier defines an opening on the front side, one or more apertures on the back side distal to the opening and positioned between the first end region and the second end region, and a chamber within the fluid collection device. At least one of the first end region or the second end region may be substantially planar. The fluid permeable body is positioned within the chamber and extends across at least a portion of the opening.

### CROSS-REFERENCE TO RELATED APPLICATIONS

This application is a continuation of U.S. patent application Ser. No. 17/657,474 filed Mar. 31, 2022, which claims priority to U.S. Provisional Patent Application No. 63/171,165 filed on Apr. 6, 2021, the disclosure of each of which is incorporated herein, in their entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to fluid collection devices, and related systems and methods. In an embodiment, a fluid collection system includes a fluid collection device having a fluid impermeable barrier and a fluid permeable body. The fluid impermeable barrier includes a first end region, a second end region, a front side, and a back side. The fluid impermeable barrier defines an opening on the front side, one or more apertures on the back side distal to the opening and positioned between the first end region and the second end region, and a chamber within the fluid collection device. The fluid permeable body is positioned within the chamber and extends across at least a portion of the opening.

In an embodiment, a method of collecting fluid includes positioning an opening defined by a fluid impermeable barrier of a fluid collection device at least proximate to a urethral opening of a user with a fluid permeable body extending at least partially across the opening being positioned at least proximate to the urethral opening. The method also includes collecting fluid discharged by the user in a chamber of the fluid impermeable barrier. The method also includes passing the fluid in the chamber through one or more apertures on a back side of the fluid impermeable barrier distal to the opening and positioned between a first end region and a second end region of the fluid impermeable barrier.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A front isometric view of a fluid collection device, according to an embodiment.

FIG. 1B is a side view of the fluid collection device of FIG. 1A, according to an embodiment.

FIG. 1C is a cross-sectional view of the fluid collection device of FIG. 1A taken along line 1C-1C of FIG. 1A.

FIG. 2A is a cross-sectional view of a portion of a fluid permeable body, according to an embodiment.

FIG. 2B is a cross-sectional view of a portion of a fluid permeable body, according to an embodiment.

FIG. 3A is a side view of a fluid collection device, according to an embodiment.

FIG. 3B is a block diagram of a fluid collection system, according to an embodiment.

FIG. 4A is a front view of an undergarment for securing a fluid collection device to a user, according to an embodiment.

FIG. 4B is a front view of an undergarment for securing a fluid collection device to a user, according to an embodiment.

FIG. 5 is a flow diagram of a method for collecting fluid, according to an embodiment.

### DETAILED DESCRIPTION

Embodiments disclosed herein are related to fluid collection devices having one or more openings on a back side of the fluid collection device, and related systems and methods. In at least one, some, or all embodiments of fluid collection devices described herein, the fluid collection devices includes at least one substantially planar end region, thereby providing a more flat fluid collection device that may provide a more comfortable use of the fluid collection device by patients while sitting. In some embodiments, the fluid collection devices may include a spine (e.g., support member) included in the fluid collection device for approximately half of a longitudinal length of the fluid collection device. In at least one, some, or all embodiments of fluid collection devices described herein, the spine may allow the fluid collection device to form and retain a desired shape with a portion of the fluid collection device positioned against a urethral opening of the user and a portion of the fluid collection device positioned underneath the user.

The fluid collection devices may include a back side that is permeable and/or includes one or more apertures (e.g. holes or openings) sized and positioned to allow fluid to pass through the backside. In some embodiments, the fluid collection devices may include a fluid impermeable collection bag secured to the fluid collection device and positioned to receive urine discharged in the fluid collection device through the one or more apertures on the back side of the fluid collection device. In use, once the chamber defined at least partially by the fluid impermeable is filled to at least partial capacity, fluid may flow from the chamber, through the one or more apertures, and into an interior region of the fluid impermeable collection bag, thereby giving the fluid collection device more capacity for fluid collection. The fluid impermeable collection bag may be sized and dimensioned to be positioned between legs of the user and may be flexible to be positioned in whatever space may be available between the legs of the user. Fluid collection devices described herein may be of particular use to individuals sitting in a wheelchair. The fluid impermeable collection bag may be supported on the seat of the chair between the legs of the user. The interior region of the fluid impermeable collection bag may include at least one of an odor elimination agent (such as vitamin C) or a solidifying agent (such as gel beads).

In at least one, some, or all embodiments of fluid collection devices described herein, the fluid collection device having a fluid impermeable collection bag secured to the fluid impermeable barrier may eliminate the need to store and utilize a pump that draws fluid from the fluid collection device. In at least one, some, or all embodiments of fluid collection device described herein, the fluid impermeable collection bag allows individuals having incontinence issues that result in small, gradual fluid discharge throughout the day to use the fluid collection device.

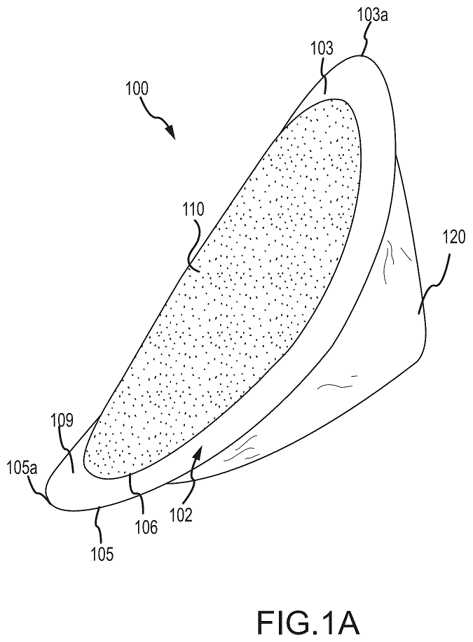

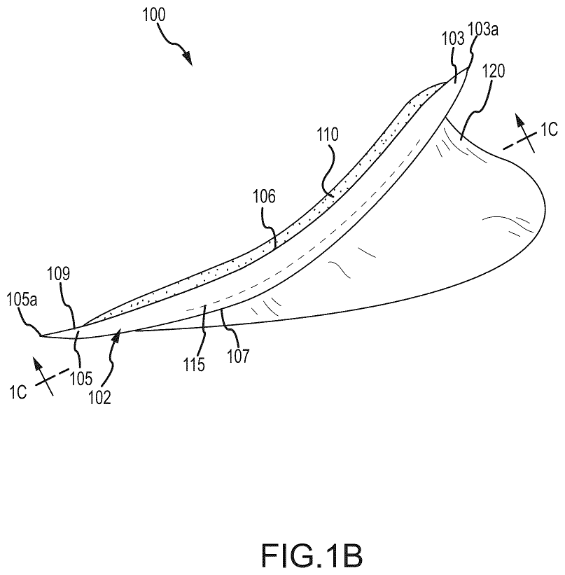

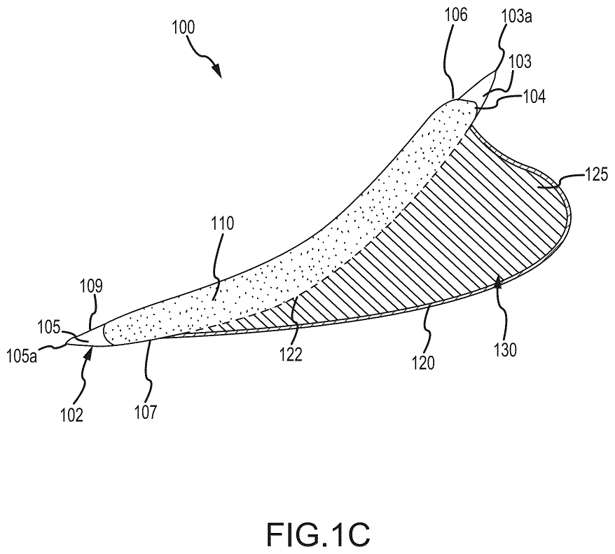

FIG. 1A is a side isometric view of a fluid collection device having a fluid impermeable collection bag 120, FIG. 1B is a side view of the fluid collection device 100, and FIG. 1C is a cross-sectional view of the fluid collection device taken along line 1C-1C of FIG. 1B. The fluid collection device 100 is an example of a fluid collection device 100 that is configured to receive fluids from a female or a male, such as a male having a buried penis. In at least one, some, or all embodiments of the fluid collection device 100, the configuration of the fluid collection device 100 may draw urine or other fluids away from the urethral opening and/or skin of the individual for collection in the fluid impermeable collection bag 120 without assistance from a vacuum and/or gravity. The fluid collection device 100 includes a fluid impermeable barrier 102 having a distal end region 105, a proximal end region 103, and a front side 109 at least partially defining an opening 106, according to an embodiment. The fluid collection device 100 also includes a back side 107 defining one or more apertures 122 substantially opposite to the opening 106. The fluid impermeable barrier 102 may at least partially define a chamber 104 (e.g., interior region, shown in FIG. 1C) and include an inward border or edge defining an opening 106 on the front side 109. A fluid permeable body 110 may be positioned within the chamber 104 and extend across at least a portion (e.g., all) of the opening 106.

The fluid impermeable barrier 102 may be elongated and may include a substantially flat or planar edge 103a, 105a or rim at one or more of the distal end region 105 or the proximal end region 103. The fluid impermeable barrier 102 may include a substantially oval or half-oval front profile, or other suitable profile. In some embodiments, the fluid impermeable barrier 102 and/or the fluid permeable body 110 may be contoured to protrude or arc relative to the substantially flat or planar edge 103a, 105a, giving the fluid collection device 100 a “duck-bill” shape. This shape of the fluid collection device 100 may be more comfortable to a user while the user is sitting. The substantially flat edge 103a, 105a, for example, may be comfortable for the user when positioned under the user. In some embodiments, the fluid impermeable barrier 102 and the fluid permeable body may be substantially cylindrical.

The opening 106 is formed in and extends longitudinally through the front side 109 the fluid impermeable barrier 102, thereby enabling fluids to enter the chamber 104 from outside of the fluid collection device 100. The opening 106 may be configured to be positioned at least proximate to (e.g., adjacent to) the opening of a female urethra or over the opening of a urethra of a male having a buried penis. The opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge of the fluid impermeable barrier 102 defines the opening 106. The edge may include two opposing arced portions, the arc portions following the outer circumference or periphery of the fluid impermeable barrier 102. The region of the back side 107 defining the one or more apertures 122 may be fluid impermeable and may include the same or different material as the fluid impermeable barrier 102.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone. In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing.

Turning to FIG. 1C, the fluid collection device 100 may include a back side 107 defining one or more apertures 122 that provides fluid communication between the chamber 104 and an interior region 125 of the fluid impermeable collection bag 120. The region of the back side 107 defining the one or more apertures 122 may include any of the materials described above in relation to the fluid impermeable barrier 102, and the one or more apertures 122 may include a plurality of apertures (e.g., holes or openings) in the back side 107 sized to allow fluid to pass from the chamber 104, through the plurality of apertures 122, and into the interior region 125 of the fluid impermeable collection bag 120. In some embodiments, the region of the back side 107 defining the one or more apertures 122 may include a material different than the fluid impermeable barrier 102. For example, the region of the back side 107 defining the one or more apertures 122 may include a fluid permeable material or membrane positioned between the interior region 125 and the chamber 104. The region of the back size 107 defining the plurality of apertures 122 may include a one-way material or fabric that allows fluid to pass through the one-way material or fabric into the interior region 125 from the chamber 104, but prevents fluid from flowing through the one-way material or fabric into the chamber 104 from the interior region 125.

The region of the back side 107 defining the plurality of apertures 122 may extend at least partially between the proximal end region 103 and the distal end region 105. For example the plurality of apertures 122 may be positioned from the proximal end region 103 to at least about one-quarter of a longitudinal length to the distal end region 105, at least about one-third of the longitudinal length to the distal end region 105, at least about one-half of the longitudinal length to the distal end region 105, at least about two-thirds of the longitudinal length to the distal end region 105, at least about three-quarters of the longitudinal length to the distal end region 105, about one-quarter of the longitudinal length to the distal end region 105, about one-third of the longitudinal length to the distal end region 105, about one-half of the longitudinal length to the distal end region 105, about two-thirds of the longitudinal length to the distal end region 105, about three-quarters of the longitudinal length to the distal end region 105, or substantially all of the longitudinal length to the distal end region 105. The plurality of apertures 122 may be positioned to extend across a portion of a lateral width of the back side 107, such as about one-eighth, about one-quarter, about one-third, about one-half, about two-thirds, about three-quarters, or substantially all of a lateral width of the back side 107.

The fluid impermeable collection bag 120 may be secured or securable to the fluid impermeable barrier 102 to cover the plurality of apertures 122. The fluid impermeable collection bag 120 may include any of the materials described above in relation to the fluid impermeable barrier 102. In some embodiments, the fluid impermeable collection bag 120 may include a fluid impermeable film that imparts the bag-like characteristic of the fluid impermeable collection bag 120. In some embodiments, the fluid impermeable collection bag 120 includes one or more of polyvinyl chloride and phthalates, polypropylene, ethylene vinyl acetate, or combinations thereof. The fluid impermeable collection bag 120 may be flexible to form whatever space is available between legs of the user. In some embodiments, the fluid impermeable collection bag 120 may include any suitable flexible or rigid container that is secured or securable to the fluid impermeable barrier 102. In some embodiments, the fluid impermeable collection bag 120 is fixedly secured to the fluid impermeable barrier 102. In some embodiments, the fluid impermeable collection bag 120 is selectively and/or removably securable to the fluid impermeable barrier 102, and the fluid impermeable barrier 102 may be re-usable with a different or new fluid impermeable collection bag 120 after a previous fluid impermeable collection bag 120 has been use (e.g., at least partially filled with fluid).

As previously discussed, the fluid collection device 100 also includes the fluid permeable body 110 positioned within the chamber 104 and extending at least partially across the opening 106 and at least partially between the distal end region 105 and the proximal end region 103. The fluid permeable body 110 may be shaped generally complementary to the shape of the chamber 104 of the fluid impermeable barrier 102. In some embodiments, the fluid permeable body 110 fills substantially all of the chamber 104.

The fluid permeable body 110 can be configured to wick and/or allow transport of fluid away from the opening 106 towards the plurality of apertures 122. The fluid permeable body 110 can include any material that can wick and/or allow transport of the fluid. The permeable properties referred to herein can be wicking, capillary action, diffusion, or other similar properties or processes, and are referred to herein as “permeable” and/or “wicking.” Such “wicking” or other physical properties may exclude absorption into the fluid permeable body 110, such as not include adsorption of the bodily fluids into the fluid permeable body 110. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 110 (e.g., absorbency), such as less than about 30 wt % of the dry weight of the fluid permeable body 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 110. In an embodiment, the fluid permeable body 110 may include at least one absorbent or adsorbent material.

The fluid permeable body 110 can include a one-way fluid movement fabric. As such, the fluid permeable body 110 can remove fluid from the area around the penis, thereby leaving the area and urethra dry. The fluid permeable body 110 can enable the fluid to flow generally towards the plurality of apertures 122. The fluid permeable body 110 can include a porous or fibrous material, such as hydrophilic polyolefin. In some embodiments, the fluid permeable body 110 consists of or consists essentially of a porous or fibrous material, such as hydrophilic polyolefin. Examples of polyolefin that can be used in the fluid permeable body 110 include, but are not limited to, polyethylene, polypropylene, polyisobutylene, ethylene propylene rubber, ethylene propylene diene monomer, or combinations thereof. Moreover, the fluid permeable body 110 can be manufactured according to various manufacturing methods, such as molding, extrusion, or sintering. The fluid permeable body 110 can include varying densities or dimensions.

In some embodiments, the fluid permeable body 110 can include two or more layers of fluid permeable materials. For example, the fluid permeable body 110 can include a fluid permeable membrane covering or wrapped around a fluid permeable support, with both the fluid permeable membrane and the fluid permeable support being disposed in the chamber 104. The fluid permeable membrane can cover or extend across at least a portion (e.g., all) of at least the side of the fluid permeable support facing the penis of the user. The fluid permeable membrane and the fluid permeable support can be configured to wick any fluid away from the opening 106, thereby preventing the fluid from escaping the chamber 104 through the opening 106 and promoting removal of the fluid from the chamber 104 through the plurality of apertures 122. The permeable properties referred to herein can be wicking, capillary action, diffusion, or other similar properties or processes, and are referred to herein as “permeable” and/or “wicking.”

The fluid permeable membrane and the fluid permeable support also can wick and/or allow transport of the fluid generally towards the plurality of apertures 122. The fluid permeable membrane can include any material that can wick the fluid. For example, the fluid permeable membrane can include fabric, such as a gauze (e.g., a silk, linen, polymer based materials such as polyester, or cotton gauze), nylon (such as a spun nylon fibers), another soft fabric (e.g., jersey knit fabric or the like), or another smooth fabric (e.g., rayon, satin, or the like). Forming the fluid permeable membrane from gauze, soft fabric, and/or smooth fabric can reduce chaffing caused by the fluid collection device 100. Other embodiments of fluid permeable membranes and fluid permeable supports are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 110 includes a fluid permeable support including a porous spun nylon fiber structure and a fluid permeable wicking membrane including gauze at least partially enclosing the spun nylon fiber structure. For example, the fluid permeable body 110 may include a gauze or other wicking fabric positioned to contact the skin of the user through the opening 106. In some embodiments, the gauze or other wicking fabric is wrapped around a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

In some embodiments, the fluid collection device 100 also may include an additional fluid permeable body 130 positioned within the interior region 125 of the fluid impermeable collection bag 120. The additional fluid permeable body 130 may be configured to wick or draw fluid away from the plurality of apertures 122 into the interior region 125 of the fluid impermeable collection bag 125. In some embodiments, the additional fluid permeable body 130 may include any of the materials described above in relation to the fluid permeable body 110. The additional fluid permeable body 130 may cover at least a portion (e.g., all) of the plurality of apertures 122. In some embodiments, the additional fluid permeable body 130 may be adjacent to the plurality of apertures 122 and/or interfacing the region of the back side 107 defining the plurality of apertures 122. For example, the additional fluid permeable body 130 may extend from the region of the back side 107 defining the plurality of apertures 122 into the interior region 125 of the fluid impermeable collection bag 120. In some embodiments, the additional fluid permeable body 120 fills at least a portion (e.g., all) of the interior region 125 of the fluid impermeable collection bag 120. In some embodiments, the additional fluid permeable body 120 extends from the plurality of apertures to the back of the fluid impermeable collection bag 120.

In use, in some conditions, the fluid permeable body 110 swells and capillaries within the fluid permeable body 110 are blocked, the wicking properties of the fluid collection device 100 are lowered or reduced. Thus, without a pump or vacuum source, the fluid collection device 100 may last longer because the urine can be transferred from the chamber 104 to the interior region 125 of the fluid impermeable collection bag 120. Transfer of the fluid from the chamber 104 through the plurality of apertures 122 and into the interior region 125 of the fluid impermeable collection bag 120 may happen mechanically (e.g., squeezing the fluid impermeable barrier 102) and/or through hydrophilic material properties, described in greater detail below. The interior region 125 of the fluid impermeable collection bag 120 may include at least one of an odor elimination agent (such as vitamin C) or a solidifying agent (such as gel beads). In some embodiments, the additional fluid permeable body 130 may be absent and the interior region 125 is open space void of other materials.

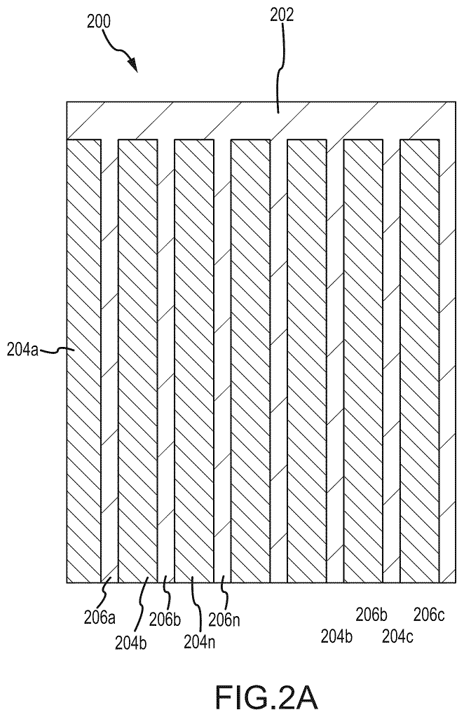

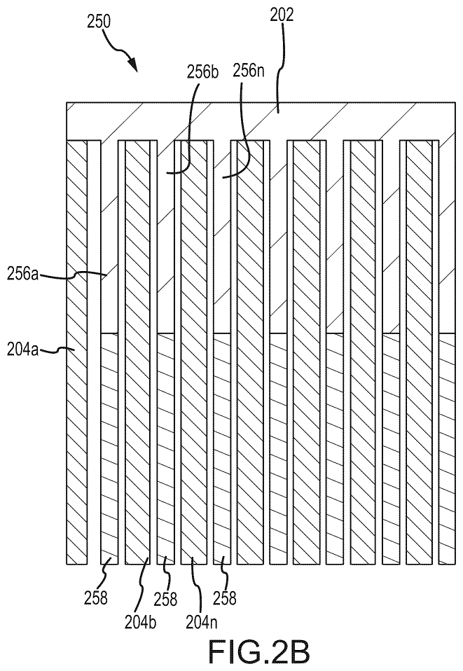

FIG. 2A is a cross-sectional view of a portion of an embodiment of an additional fluid permeable body 200 that may be positioned within the interior region 125 of the fluid impermeable collection bag 120 (not shown). Unless otherwise noted, the additional fluid permeable body 200 may include any aspect the additional fluid permeable body 120, such as size, positioning within the interior region 125 of the fluid impermeable collection bag 120, and so forth. In some embodiments, to prevent capillary blocking in the fluid permeable body 110 that slows down the urine transfer away from the skin, the additional fluid permeable body 200 may include a structured combination of fast wicking fibers and high absorbent material to conduct the urine output quickly can be used. For example, the additional fluid permeable body 200 may include at least a region of wicking material 202 positioned adjacent to (e.g., covering or otherwise in fluid communication with) the plurality of apertures 122 in the back side 107. The wicking material 202 may include any of the wicking materials and characteristics described above in relation to the wicking material of the fluid permeable body 110. In some embodiments, the wicking material 202 may extend across at least a portion (e.g., all) of the plurality of apertures 122. The wicking material 202 may extend from the proximal end region 103 to at least about one-quarter of a longitudinal length to the distal end region 105, at least about one-third of the longitudinal length to the distal end region 105, at least about one-half of the longitudinal length to the distal end region 105, at least about two-thirds of the longitudinal length to the distal end region 105, at least about three-quarters of the longitudinal length to the distal end region 105, about one-quarter of the longitudinal length to the distal end region 105, about one-third of the longitudinal length to the distal end region 105, about one-half of the longitudinal length to the distal end region 105, about two-thirds of the longitudinal length to the distal end region 105, about three-quarters of the longitudinal length to the distal end region 105, or substantially all of the longitudinal length to the distal end region 105.

The additional fluid permeable body 200 also may include strips, sheets, columns, or cylinders of wicking material 206 and strips, sheets, columns, or cylinders of absorbent material 204 extending from the wicking material 202. The region of wicking material 202 and/or the strips of wicking material 206 may include one or more of bamboo fiber, cotton fiber, hemp fiber, microfiber, or combinations thereof. The strips of absorbent material 204 may include sodium polyacrylate, cross-linked co-polymers of acrylamide and potassium acrylate, reclaimed fluff pulp, or other absorbent material(s) can be used as well.

In some embodiments, the strips of wicking material 206 and the strips of absorbent material 204 may be oriented in a direction of the flow of fluid in the additional fluid permeable body 200 due to gravity. The strips of wicking material 206 and strips of absorbent material 204 may be arranged in an alternating configuration such that each strip of wicking material is positioned adjacent to one or two strips of absorbent material 204, and each strip of absorbent material 204 is positioned adjacent to one or two strips of wicking material 206. For example, the additional fluid permeable body 200 may include a first strip 204a of absorbent material extending from the region of wicking material 202, a first strip 206a of additional wicking material extending from the region of wicking material 202 and positioned adjacent to the first strip 204a of absorbent material, a second strip 204b of absorbent material extending from the region of wicking material 202 and positioned adjacent to the first strip 206a of additional wicking material such that the first strip 206a of additional wicking material is positioned between the first strip 204a of absorbent material and the second strip 204b of absorbent material, and a second strip 206b of additional wicking material extending from the region of wicking material 202 and positioned adjacent to the second strip 204b of absorbent material such that the second strip of absorbent material 204b is positioned between the first strip 206a of additional wicking material and the second strip 206b of additional wicking material. The strips 206n of additional wicking material and the strips 204n of absorbent material continue this alternating arrangement. In some embodiments, the additional fluid permeable body 210 may include a grid of rows and columns each including alternating strips of absorbent material and strips of wicking material. The orientation of the strips of wicking material 206 and the strips of absorbent material 204 speed up the urine transportation from the chamber 104 of the fluid impermeable barrier 102 into the interior region 125 of the fluid impermeable collection bag 120 to prevent flooding or overflow of the fluid collection device 100. In such a structured combination, the strips of absorbent material 204 will not form a continuous gel layer that would slow down the speed of transfer of urine into the interior region 125 of the fluid impermeable collection bag 120. Performance of the fluid collection device 100 may be improved as the additional fluid permeable body 200 continues to function until full saturation. The strips of wicking material 206 may interface or be spaced from adjacent strips of the strips of absorbent material 204.

FIG. 2B is a cross-sectional view of a portion of an embodiment of an additional fluid permeable body 250 that may be positioned within the interior region 125 of the fluid impermeable collection bag 120 (not shown). Unless otherwise noted, the additional fluid permeable body 250 may include any aspect the additional fluid permeable body 120, 250, such as size, positioning within the interior region 125 of the fluid impermeable collection bag 120, and so forth. The additional fluid permeable body 250 may include the region of wicking material 202 and strips of absorbent material 204 positioned as described above in relation to the fluid permeable body 200.

The additional fluid permeable body 250 may include strips of additional wicking material 256, such as strips 256a, 256b, 256n. Unless otherwise noted, the strips of additional wicking material 256 include any aspect of the strips of additional wicking material 206, including material and/or positioning relative to the strips of absorbent material 204. In some embodiments, the strips of wicking material 256 may include a hydrophilic coating 258 on the strips of wicking material 256 that create a gradient in hydrophilicity that increases along direction of the flow of fluid due to gravity. This gradient in hydrophilicity may provide a continued, capillary draw for the fluid to flow.

Returning to FIG. 1B, in the illustrated embodiment, the fluid collection device 100 also may include a support member 115 secured to the fluid collection device 100. However, in other embodiments, the support member 115 may be omitted. The support member 115 may be embedded in the fluid impermeable barrier 102, embedded in the fluid permeable body 110, or positioned between the fluid permeable body 110 and the fluid impermeable barrier 102. The support member 115 may extend at least partially between the proximal end region 103 and the distal end region 105. In some embodiments the support member 115 extends from the proximal end region 103 at least partway to the distal end region 105. For example, the support member 115 may extend from the proximal end region 103 about one-half, about two-thirds, about three-quarters, at least about one-quarter, at least about one-half, at least about two-thirds, or at least about three-quarters of a distance to the distal end region 105.

The support member 115 is configured to bend or manipulate, and retain the shape manipulated to after manipulation. This support member 115, then, provides a more controllable fluid collection device 100 that can be manipulated into at least a partially compact configuration. For example, the support member 115 may be configured to be manipulated to a generally planar shape and retain the generally planar shape, thereby supporting the back side 107 of the fluid impermeable barrier 205 along an imaginary or theoretical plane. The support member 115 also may be configured to be manipulated to a generally curved, contoured, or arced shape and retain the generally curved or arced shape, thereby supporting the back side 109 of the fluid impermeable barrier 102 in a generally curved, contoured, or arced shape (similar to that of the fluid collection device 100 shown in FIG. 1A).

The support member 115 may include a shape memory material, such as a shape memory polymer or a metal (e.g., shape memory metal). Suitable shape memory materials are composed to adopt an intermediate or permanent shape in response to a stimuli. The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal wire bent to a first shape may be utilized as the shape memory material, whereinafter the metal wire may be modified to a second shape via physical force applied thereto or via heating.

In an embodiment, the shape memory material may include metal, such as an elemental metal, an alloy, or shape memory alloy. Suitable shape memory metals may include standard steels, stainless steel, carbon alloy steel, head treated steel, aluminum, silver, copper, iron, nickel, zinc, tin, beryllium, or the like. Suitable shape memory alloys may include stainless steel; galvanized steel; aluminum alloys; nickel-titanium alloys, such as Nitinol, Ni—Ti—Cu, Ni—Ti, Co, or the like; copper-based alloys such as Cu—Zn—Al, Cu—Al—Ni, Cu—Al—Sn, or the like; Co—Cr—Ni—Mo alloys (e.g., Elgiloy®) or the like; or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly(¿-caprolactone), polyethyleneterephthalate (PET), polyethyleneoxide (PEO), polyethylene glycol (PEG), polystyrene, polymethylmethacrylate (PMMA), Polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polysulphone, or the like; Polynorbonene; other deformable polymers; or any other shape memory polymer.

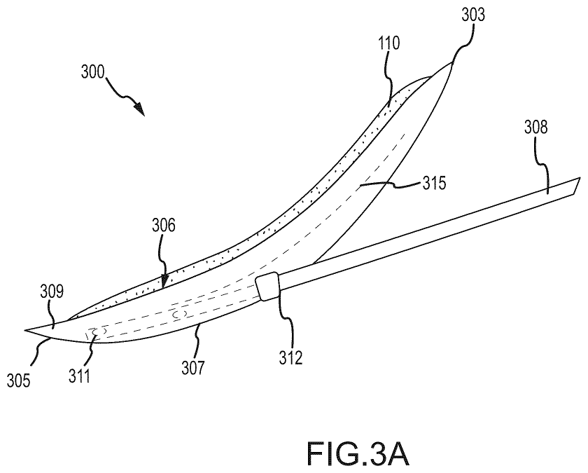

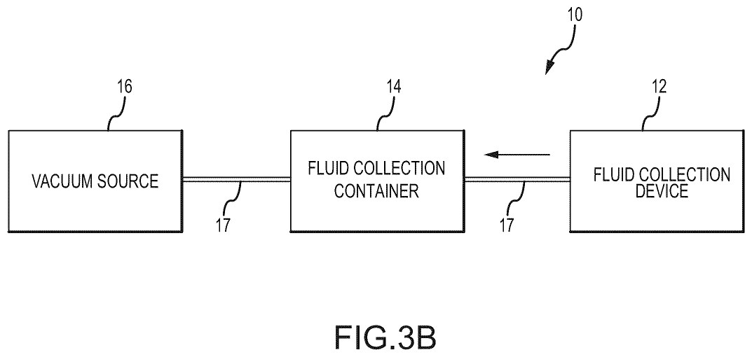

Turning now to FIG. 3A, a fluid collection device 300 may include a fluid impermeable barrier 302 and the fluid permeable body 110. Unless otherwise noted, the fluid collection device 300 may include any aspect of the fluid collection device 100. For example, the fluid impermeable barrier 302 may include a proximal end region 303, a distal end region 305, a front side 309, and a back side 307. The fluid impermeable barrier may define an opening 306 on the front side 309. The fluid permeable body 110 is positioned within the chamber and extends across at least a portion of the opening 306. The fluid collection device 315 also may include a support member 315.

The fluid collection device 300 also may include an aperture 312 or port positioned on the back side 307 of the fluid impermeable barrier 302 between the proximal end region 303 and the distal end region 305. The aperture 312 may be sized to allow a conduit 308 (e.g., tube) to slide or otherwise extend therethrough. The conduit 308 extends through the aperture 312, a seal is formed around the conduit 308 that prevents fluid from leaking through the aperture 312. The aperture 312 may be positioned approximately halfway between the proximal end region 303 and the distal end region 305.

The conduit 308 may extend through the aperture 312 into the chamber and at least proximate to the distal end region 305. The conduit 308 may include at least one opening or inlet at a terminating end of the conduit 308 positioned within the chamber. In some embodiments, the conduit 308 includes multiple openings 311 positioned in the chamber between the terminating end and the aperture 312. The openings 311 in the conduit 308 provide fluid communication between the chamber and the conduit 308. The conduit 308 may be configured to provide fluid communication between a vacuum source and the chamber of the fluid collection device 312 to draw fluid from the chamber and through the conduit 308 for collection.

FIG. 3B is a block diagram of a fluid collection system 10 that may be used with the fluid collection device 300 or other fluid collection devices described herein, according to an embodiment. The fluid collection system 10 may be included in any of the embodiments of fluid collection systems described herein. The system 10 includes a fluid (e.g., urine) collection device 12 (e.g., any of the fluid collection assemblies disclosed herein), a fluid collection container 14, and a vacuum source 16 (e.g., pump). The fluid collection device 12, the fluid collection container 14, and the vacuum source 16 may be fluidly coupled to each other via one or more tubes 17 or conduits. For example, fluid collection device 12 may be operably coupled to one or more of the fluid collection container 14 or the vacuum source 16 via the tube 17. In some embodiments, the vacuum source 16 may be secured directly to the fluid collection container 14. Fluid (e.g., urine or other bodily fluids) collected in the fluid collection device 12 may be removed from the fluid collection device 12 via the tube 17 secured to the fluid collection device 12. Suction force may be introduced into the chamber of the fluid collection device 12 via the inlet of the tube 17 responsive to suction (e.g., vacuum) force applied at the outlet of the tube 17.

The suction force may be applied to the outlet of the tube 17 by the vacuum source 16 either directly or indirectly. The suction force may be applied indirectly via the fluid collection container 14. For example, the outlet of the tube 17 may be disposed within or fluidly coupled to an interior region of the fluid collection container 14 and an additional tube 17 may extend from the fluid collection container 14 to the vacuum source 16. Accordingly, the vacuum 16 may apply suction to the fluid collection device 12 via the fluid collection container 14. The suction force may be applied directly via the vacuum source 16. For example, the outlet of the tube 17 may be disposed within the vacuum source 16. An additional tube 17 may extend from the vacuum source 16 to a point outside of the fluid collection device 12, such as to the fluid collection container 14. In such examples, the vacuum source 16 may be disposed between the fluid collection device 12 and the fluid collection container 14.

The fluid collection container 14 is sized and shaped to retain a fluid therein. The fluid collection container 14 may include a bag (e.g., drainage bag), a bottle or cup (e.g., collection jar), or any other enclosed container for storing bodily fluid(s) such as urine. In some examples, the tube 17 may extend from the fluid collection device 12 and attach to the fluid collection container 14 at a first point therein. An additional tube 17 may attach to the fluid collection container 14 at a second point thereon and may extend and attach to the vacuum source 16. Accordingly, a vacuum (e.g., suction) may be drawn through fluid collection device 12 via the fluid collection container 14. Fluid, such as urine, may be drained from the fluid collection device 12 using the vacuum source 16.

The vacuum source 16 may include one or more of a manual vacuum pump, and electric vacuum pump, a diaphragm pump, a centrifugal pump, a displacement pump, a magnetically driven pump, a peristaltic pump, or any source configured to produce a vacuum (e.g., negative pressure). The vacuum source 16 may provide a vacuum or suction to remove fluid from the fluid collection device 12. In some examples, the vacuum source 16 may be powered by one or more of a power cord (e.g., connected to a power socket), one or more batteries, or even manual power (e.g., a hand operated vacuum pump). In some examples, the vacuum source 16 may be sized and shaped to fit outside of, on, or within the fluid collection device 12. For example, the vacuum source 16 may include one or more miniaturized pumps or one or more micro pumps. The vacuum sources disclosed herein may include one or more of a switch, a button, a plug, a remote, or any other device suitable to activate the vacuum source 16. Aspects of the fluid collection system 10 may be included in any of the fluid collection systems described herein. For example, the fluid collection device 12 may include any of the fluid or urine collection devices described herein.

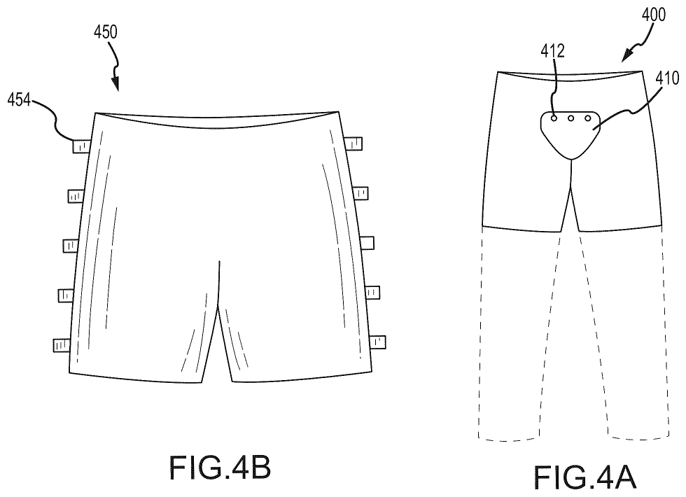

Turning to FIG. 4A, a fluid collection system may include an undergarment 400 and a fluid collection device 100, 300. The undergarment 400 may be configured to retain the fluid collection device 100, 300 in position on the user with the fluid permeable body 110 positioned at least proximate to the urethra of the user while the user is sitting in a wheelchair or other chair. In some embodiments, the undergarment 400 may include a flap 410 configured to hold the fluid collection device 100, 300 in position. The undergarment 400 may include one or more securement elements 412 configured to selectively hold the flap in a closed position that holds the fluid collection device 100, 300 in position. The one or more securement elements 412 may include hook and loop fastener material, adhesives, buttons, pins, and so forth.

Turning to FIG. 4B, an undergarment 450 may be configured to secure to a user sitting in a wheelchair. Although not shown in FIG. 4B, the undergarment 450 may include the flap 410 of the undergarment 400. The undergarment 450 may include a front side, a back side, and one or more side securement elements 454 configured to selectively secure the front side of the undergarment 450 to the back side of the undergarment 450. The one or more side securement elements 454 may include hook and loop fastener material, adhesives, buttons, pins, and so forth. The undergarments 400, 450 are configured to allow removal and replacement of the fluid collection device 100, 300 without having to fully remove the undergarments 400, 450.

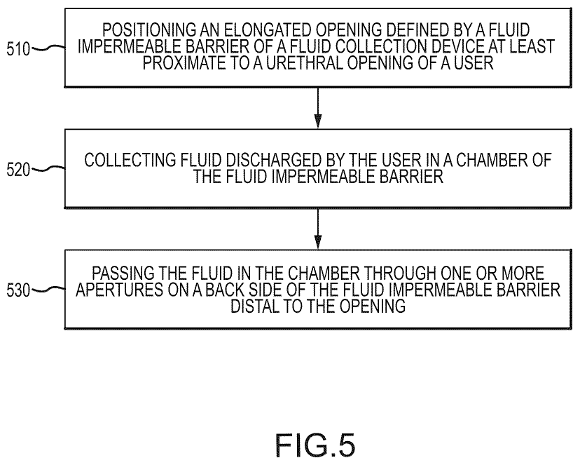

FIG. 5 is a flow diagram of a method 500 for securing a fluid collection device on a user, according to an embodiment. The method 500 includes an act 510 of positioning an opening defined by a fluid impermeable barrier of a fluid collection device at least proximate to a urethral opening of a user with a fluid permeable body extending at least partially across the opening being positioned at least proximate to the urethral opening. The method also includes an act 520 of collecting fluid discharged by the user in a chamber of the fluid impermeable barrier. The method also includes an act 530 of passing the fluid in the chamber through one or more apertures on a back side of the fluid impermeable barrier distal to the opening and positioned between a first end region and a second end region of the fluid impermeable barrier.

In some embodiments, the act 530 of passing the fluid in the chamber through one or more apertures on a back side of the fluid impermeable barrier may include passing the fluid in the chamber through a plurality of apertures on the back side of the fluid impermeable barrier into an interior region of a fluid impermeable collection bag secured to the fluid impermeable barrier. More particularly, passing the fluid in the chamber through a plurality of apertures on the back side of the fluid impermeable barrier into a fluid impermeable collection bag secured to the fluid impermeable barrier may include squeezing the fluid impermeable barrier effective to force the fluid in the chamber through the plurality of apertures into the interior region of the fluid impermeable collection bag.

In some embodiments, the method 500 may include an act of drawing the fluid in the interior region of the fluid impermeable collection bag away from the plurality of openings with an additional fluid permeable body including at least a region of wicking material positioned adjacent to the plurality of apertures in the fluid impermeable barrier. More particularly, the method may include drawing the fluid in the interior region of the fluid impermeable collection bag away from the plurality of openings with an additional fluid impermeable barrier including the region of wicking material positioned adjacent to the one or more apertures, a first of strip of absorbent material extending from the region of wicking material, a first strip of additional wicking material extending from the region of wicking material and positioned adjacent to the first strip of absorbent material, a second strip of absorbent material extending from the region of wicking material and positioned adjacent to the first strip of additional wicking material such that the first strip of additional wicking material is positioned between the first strip of absorbent material and the second strip of absorbent material, and a second strip of additional wicking material extending from the region of wicking material and positioned adjacent to the second strip of absorbent material such that the second strip of absorbent material is positioned between the first strip of additional wicking material and the second strip of additional wicking material. Each of the first strip of additional wicking material and the second strip of additional wicking material may include a hydrophilic coating on a distal region that is distal to the region of wicking material.

In some embodiments, the act 530 of passing the fluid in the chamber through one or more apertures on a back side of the fluid impermeable barrier may include passing the fluid through a conduit extending through one aperture, the conduit extending at least partially into the chamber.

The method 500 may further include an act of bending the fluid collection device in a curve or contour complementary to user. The fluid collection device may include a support member secured to the fluid collection device and configured to retain the fluid collection device in the curve complementary to the user. The method 500 may further include an act of holding the fluid collection device against the user with a flap of an undergarment held in a closed position with one or more securement elements. The method 500 may further comprise an act of securing the undergarment to the user with one or more side securement elements securing a front side of the undergarment to a back side of the undergarment.

The acts of the method 500 described above are for illustrative purposes. For example, the acts of the method 500 can be performed in different orders, split into multiple acts, modified, supplemented, or combined. In an embodiment, one or more of the acts of the method 500 can be omitted from the method 500. Any of the acts of the method 500 can include using any of the fluid collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A fluid collection device, comprising:
a fluid impermeable barrier having a distal end region, a proximal end region, a front side, and a back side, the fluid impermeable barrier defining an opening on the front side, one or more apertures on the back side spaced from the opening and positioned between the distal end region and the proximal end region, and a chamber within the fluid collection device, wherein the fluid impermeable barrier tapers between the front side and the back side forming a proximal edge at the proximal end region and a distal edge at the distal end region where the front side and the back side meet; and
a fluid permeable body positioned within the chamber and extending across at least a portion of the opening and the one or more apertures.

2. The fluid collection device of claim 1, wherein the fluid permeable body is contoured to protrude or arc through the opening relative to the proximal edge and the distal edge.

3. The fluid collection device of claim 2, wherein the front side of the fluid impermeable barrier includes a substantially oval periphery with the front side being substantially planar and extending inward from the substantially oval periphery to the opening defined by the front side.

4. The fluid collection device of claim 1, wherein the fluid impermeable barrier is contoured to protrude or arc relative to the proximal edge and the distal edge.

5. The fluid collection device of claim 1, further comprising:
wherein the one or more apertures include one aperture positioned on the back side of the fluid impermeable barrier between the first end region and the second end region; and
a conduit extending through the one aperture into the chamber.

6. The fluid collection device of claim 5, wherein the one aperture is positioned approximately halfway between the first end region and the second end region.

7. The fluid collection device of claim 5, wherein the conduit extends through the one aperture into the chamber and to the second end region.

8. The fluid collection device of claim 7, wherein the conduit includes a plurality of openings positioned in the chamber and in fluid communication with the chamber.

9. The fluid collection device of claim 5, further comprising a support member including a shape memory material extending longitudinally at least partially through the fluid collection device.

10. The fluid collection device of claim 9, wherein the support member extends from the proximal end region at least about two-thirds of a distance to the distal end region such that the one aperture is positioned between a proximal end of the support member and a distal end of the support member.

11. The fluid collection device of claim 1, further comprising a fluid impermeable collection bag secured to the fluid impermeable barrier and covering at least a portion of the back side of the fluid impermeable barrier, the fluid impermeable collection bag defining an interior region in fluid communication with the chamber of the fluid impermeable barrier through the one or more apertures in the back side of the fluid impermeable barrier.

12. The fluid collection device of claim 11, wherein the one or more apertures include a plurality of apertures providing fluid communication between the chamber and the interior region of the fluid impermeable collection bag.

13. The fluid collection device of claim 12, wherein the plurality of apertures are positioned in a first region of the back side of the fluid impermeable barrier that extends from at least proximate to the first end region of the fluid impermeable barrier at least halfway to the second end region of the fluid impermeable barrier.

14. The fluid collection device of claim 11, wherein the fluid collection device includes an additional fluid permeable body positioned within the interior region of the fluid impermeable collection bag, the additional fluid permeable body including at least a region of wicking material positioned adjacent to the one or more apertures in the fluid impermeable barrier.

15. The fluid collection device of claim 14, wherein the additional fluid permeable body includes:
the region of wicking material positioned adjacent to the one or more apertures;
a first strip of absorbent material extending from the region of wicking material;
a first strip of additional wicking material extending from the region of wicking material and positioned adjacent to the first strip of absorbent material;
a second strip of absorbent material extending from the region of wicking material and positioned adjacent to the first strip of additional wicking material such that the first strip of additional wicking material is positioned between the first strip of absorbent material and the second strip of absorbent material; and
a second strip of additional wicking material extending from the region of wicking material and positioned adjacent to the second strip of absorbent material such that the second strip of absorbent material is positioned between the first strip of additional wicking material and the second strip of additional wicking material.

16. The fluid collection device of claim 15, wherein the each of the first strip of additional wicking material and the second strip of additional wicking material includes a hydrophilic coating on a distal region that is distal to the region of wicking material.

17. A fluid collection system, comprising:
a fluid collection device including:
a fluid impermeable barrier having a first end region, a second end region, a front side, and a back side, the fluid impermeable barrier defining an opening on the front side, one or more apertures on the back side distal to the opening and positioned between the first end region and the second end region, and a chamber within the fluid collection device, wherein the fluid impermeable barrier tapers between the front side and the back side forming a proximal edge at the proximal end region and a distal edge at the distal end region where the front side and the back side meet; and
a fluid permeable body positioned within the chamber and extending across at least a portion of the opening; and
an undergarment having a flap configured to hold the fluid collection device against the user with the opening at least proximate to a urethral opening of the user, the undergarment including one or more securement elements configured to selectively hold the flap in a closed position.

18. The fluid collection system of claim 17, wherein the undergarment includes a front side, a back side, and one or more side securement elements configured to selectively secure the front side of the undergarment to the back side of the undergarment.

19. The fluid collection system of claim 17, wherein the fluid permeable body is contoured to protrude or arc through the opening relative to the proximal edge and the distal edge.

20. The fluid collection system of claim 19, wherein the front side of the fluid impermeable barrier includes a substantially oval periphery with the front side being substantially planar and extending inward from the substantially oval periphery to the opening defined by the front side.

21. The fluid collection system of claim 17, wherein the fluid impermeable barrier is contoured to protrude or arc relative to the proximal edge and the distal edge.

22. The fluid collection system of claim 17, further comprising:
wherein in the one or more apertures include one aperture positioned on the back side of the fluid impermeable barrier between the first end region and the second end region; and
a conduit extending through the one aperture into the chamber.

23. The fluid collection system of claim 22, further comprising a vacuum source in fluid communication with the conduit and configured to draw urine from the chamber and through the conduit.

24. The fluid collection system of claim 17, wherein the fluid collection device includes a fluid impermeable collection bag secured to the fluid impermeable barrier and covering at least a portion of the back side of the fluid impermeable barrier, the fluid impermeable collection bag defining an interior region in fluid communication with the chamber of the fluid impermeable barrier through the one or more apertures in the back side of the fluid impermeable barrier.

25. A fluid collection system, comprising:
a fluid collection device including:
a fluid impermeable barrier having a first end region, a second end region, a front side, and a back side, the fluid impermeable barrier defining an opening on the front side, an aperture on the back side distal to the opening and positioned between the first end region and the second end region, and a chamber within the fluid collection device, wherein the fluid impermeable barrier tapers between the front side and the back side forming a proximal edge at the proximal end region and a distal edge at the distal end region where the front side and the back side meet; and
a fluid permeable body positioned within the chamber and extending across at least a portion of the opening;
a conduit extending through the aperture into the chamber; and
a vacuum source in fluid communication with the conduit and configured to draw urine from the chamber and through the conduit.

26. The fluid collection system of claim 25, further comprising an undergarment having a flap configured to hold the fluid collection device against the user with the opening at least proximate to a urethral opening of the user, the undergarment including one or more securement elements configured to selectively hold the flap in a closed position.

27. The fluid collection system of claim 26, wherein the undergarment includes a front side, a back side, and one or more side securement elements configured to selectively secure the front side of the undergarment to the back side of the undergarment.

28. The fluid collection system of claim 25, wherein the fluid permeable body is contoured to protrude or arc through the opening relative to the proximal edge and the distal edge.

29. The fluid collection system of claim 25, wherein the front side of the fluid impermeable barrier includes a substantially oval periphery with the front side being substantially planar and extending inward from the substantially oval periphery to the opening defined by the front side.

30. The fluid collection system of claim 25, wherein the fluid impermeable barrier is contoured to protrude or arc relative to the proximal edge and the distal edge.
